(19) United States
(12) Patent Application Publication
(10) Pub. No.: US 2016/0143522 A1
(43) Pub. Date: May 26, 2016

(54) Visualization Catheters

(71) Applicant: LuxCath, LLC, Boston, MA (US)

(72) Inventors: Terrance J. Ransbury, Chapel Hill, NC (US); Omar Amirana, Cambridge, MA (US); Kenneth C. Armstrong, Cary, NC (US); Cinnamon Larson, Carrboro, NC (US)

(21) Appl. No.: 14/952,048

(22) Filed: Nov. 25, 2015

Related U.S. Application Data
(60) Provisional application No. 62/084,174, filed on Nov. 25, 2014.

(51) Int. Cl. A61B 1/313; A61B 1/07; A61B 1/05; A61B 18/24; A61B 1/04; A61B 18/14; A61B 18/06; A61B 18/02; A61B 1/00; A61B 8/12

(52) U.S. Cl. CPC A61B 1/3137; A61B 1/00082; A61B 1/07; A61B 1/05; A61B 8/12; A61B 1/043; A61B 18/1492; A61B 18/06; A61B 18/02; A61B 18/245; A61B 2018/1861

(57) ABSTRACT

Methods and systems for ablation mapping and ablation procedures are provided. In some embodiments, a catheter for visualizing ablated tissue comprises a catheter body; a support assembly extending past a distal end of the catheter body, the support assembly having a lumen therethrough; and a balloon having a proximal end and a distal end, wherein the proximal end of the balloon is attached to the catheter body and the distal end of the balloon is attached to the support assembly, the balloon having an opening at the distal end in alignment with the lumen of the support assembly to provide a continuous path from the catheter body to outside of the balloon.

### RELATED APPLICATIONS

This application claims the benefit of and priority to U.S. Provisional Application Ser. No. 62/084,174, filed on Nov. 25, 2014, which is incorporated herein by reference in its entirety.

### FIELD

The present disclosure generally relates to visualization catheters and systems.

### BACKGROUND

Atrial fibrillation (AF) is the most common sustained arrhythmia in the world, which currently affects millions of people. In the United States, AF is projected to affect 10 million people by the year 2050. AF is associated with increased mortality, morbidity, and an impaired quality of life, and is an independent risk factor for stroke. The substantial lifetime risk of developing AF underscores the public heath burden of the disease, which in the U.S. alone amounts to an annual treatment cost exceeding $7 billion.

Most episodes in patients with AF are known to be triggered by focal electrical activity originating from within muscle sleeves that extend into the Pulmonary Veins (PV). Atrial fibrillation may also be triggered by focal activity within the superior vena cava or other atrial structures, i.e. other cardiac tissue within the heart's conduction system. These focal triggers can also cause atrial tachycardia that is driven by reentrant electrical activity (or rotors), which may then fragment into a multitude of electrical wavelets that are characteristic of atrial fibrillation. Furthermore, prolonged AF can cause function alterations in cardiac cell membranes and these changes further perpetuate atrial fibrillation.

Radiofrequency ablation (RFA), laser ablation, and cryo ablation are the most common technologies of catheter-based mapping and ablation systems used by physicians to treat atrial fibrillation. Physician uses a catheter to direct energy to either destroy focal triggers or to form electrical isolation lines isolating the triggers from the heart's remaining conduction system. The latter technique is commonly used in what is called pulmonary vein isolation (PVI). However, the success rate of the AF ablation procedure has remained relatively stagnant with estimates of recurrence to be as high as 30% to 50% one-year post procedure.

Therefore, there is a need for a catheter for use in cardiac mapping and ablation procedures that would simplify the ablation procedures and improve success rates.

### SUMMARY

Methods and systems for ablation mapping and ablation procedures are provided. In some aspects, there is provided a catheter for visualizing ablated tissue that includes a catheter body; a support assembly extending past a distal end of the catheter body, the support assembly having a lumen therethrough; and a balloon having a proximal end and a distal end, wherein the proximal end of the balloon is attached to the catheter body and the distal end of the balloon is attached to the support assembly, the balloon having an opening at the distal end in alignment with the lumen of the support assembly to provide a continuous path from the catheter body to outside of the balloon.

In some embodiments, there is provided a system for visualizing ablated tissue that includes a catheter comprising a catheter body; a support assembly extending past a distal end of the catheter body, the support assembly having a lumen therethrough; and a balloon having a proximal end and a distal end, wherein the proximal end of the balloon is attached to the catheter body and the distal end of the balloon is attached to the support assembly, the balloon having an opening at its distal end in alignment with the lumen of the support assembly to provide a continuous path from the catheter body to outside of the balloon.

In some aspects, there is provided a method for transseptal access to a left atrium that includes advancing a catheter to a right atrium, the catheter comprising a catheter comprising a catheter body; a support assembly extending past a distal end of the catheter body, the support assembly having a lumen therethrough; a balloon having a proximal end and a distal end, wherein the proximal end of the balloon is attached to the catheter body and the distal end of the balloon is attached to the support assembly, the balloon having an opening at the distal end in alignment with the lumen of the support assembly to provide a continuous path from the catheter body to outside of the balloon; and a camera; delivering a puncturing instrument through the path in the catheter body to outside the balloon; and under visualization with the camera, pushing the puncturing instrument against the fossa ovalis to make an access hole into the left atrium.

In some aspects, there is provided a method for ablation mapping that includes advancing a catheter to a cardiac tissue in need of ablation mapping, the catheter comprising a catheter comprising a catheter body; a support assembly extending past a distal end of the catheter body, the support assembly having a lumen therethrough; a balloon having a proximal end and a distal end, wherein the proximal end of the balloon is attached to the catheter body and the distal end of the balloon is attached to the support assembly, the balloon having an opening at the distal end in alignment with the lumen of the support assembly to provide a continuous path from the catheter body to outside of the balloon; and a camera; exciting nicotinamide adenine dinucleotide hydrogen (NADH) in an area of the cardiac tissue; collecting light reflected from the cardiac tissue and directing the collected light to a light detecting instrument; imaging the area of the cardiac tissue to detect NADH fluorescence of the area of the cardiac tissue; and producing a display of the imaged, illuminated cardiac tissue, the display illustrating the ablated cardiac tissue as having less fluorescence than non-ablated cardiac tissue.

### BRIEF DESCRIPTION OF THE FIGURES

The presently disclosed embodiments will be further explained with reference to the attached drawings, wherein like structures are referred to by like numerals throughout the several views. The drawings shown are not necessarily to scale, with emphasis instead generally being placed upon illustrating the principles of the presently disclosed embodiments.

FIG. 1 is a detailed plan view of an embodiment of the balloon catheter assembly.

FIG. 2 is an oblique view of the catheter in FIG. 1 with the balloon resected to show the lumens and support assembly more clearly.

FIG. 3 is a detailed plan view of an embodiment of the balloon catheter assembly.

FIG. 4 is a detailed plan view of an embodiment of the balloon catheter assembly with the addition of an extended needle for use in certain procedures such as a cardiac transseptal puncture.

FIG. 5 is an oblique view of the catheter in FIG. 4 with the balloon resected to show the lumens and needle component more clearly.

FIG. 6 is a detailed plan view of the catheter of FIG. 4 and FIG. 5 with the needle retracted within the support assembly lumen.

FIG. 7 is an oblique view of the catheter in FIG. 4, FIG. 5 and FIG. 6 with the needle completely removed from the catheter assembly as would be the case in procedures such as a cardiac transseptal puncture.

FIG. 8A and FIG. 8B illustrate an embodiment of a balloon catheter assembly of the present disclosure.

FIG. 9A illustrates an embodiment of a diagnostic system of the present disclosure.

FIG. 9B illustrates an embodiment of a visualization system of the present disclosure.

FIG. 10 is a flow chart of an embodiment method of the present disclosure.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The present disclosure generally relates to systems and methods for imaging tissue using nicotinamide adenine dinucleotide hydrogen (NADH) fluorescence (fNADH). By way of a non-limiting example, the present systems and methods may be used in connection with cardiac mapping and ablation procedures. In some embodiments, the systems of the present disclosure may be used to assist with the treatment of Atrial Fibrillation (AF).

In some embodiments, the instant systems include a balloon visualization catheter. In some embodiments, the visualization catheter of the present disclosure facilitates illumination, in UV or other wavelength range, of a target zone and captures fluoresced light, or the lack of such light, and/or reflected light back to the physician. In some embodiments, the shape of the balloon and the balloon support assembly of the visualization catheter of the present disclosure are designed to work the ostia of pulmonary veins. However, the visualization catheter may be adapted to work anywhere in the human anatomy. For example, in some embodiments, the balloon is shaped to conform to the atrial or ventricular wall instead of the vein.

The visualization catheter 100 may have various designs depending on the particular medical application. The visualization catheter 100 can be any instrument or tool, including, but not limited to, a rigid or flexible tube, handheld surgical instrument, probe or needle-like device. In some embodiments, the visualization catheter 100 is designed for minimally invasive procedures. The visualization catheter 100 may include one or more lumens for operating the balloon 101 and for delivering energy, materials or instruments into the balloon 101 or to the site of treatment, beyond the distal end of the visualization catheter 100. The visualization catheter 100 may include a handle at its distal tip to aid in steering and generally handling the catheter 100. The handle may include one or more connections or ports in communication with the one or more lumens of the visualization catheter 100 for introducing energy, materials or instruments into the one or more lumens of the visualization catheter 100.

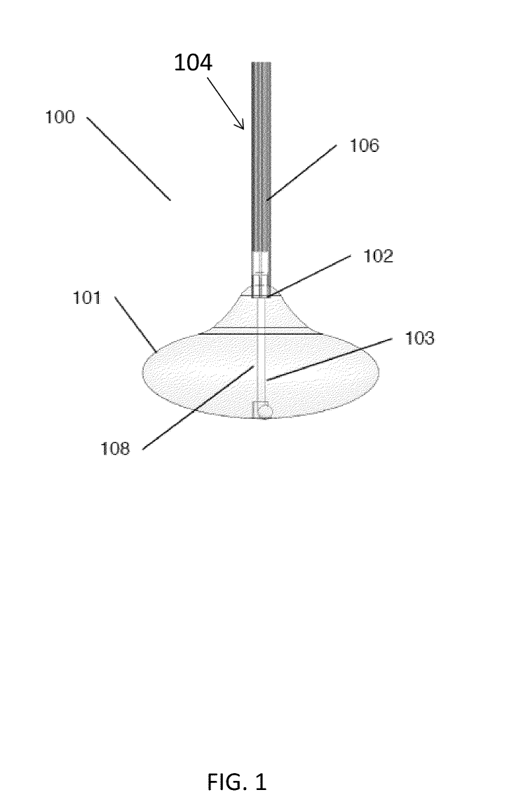

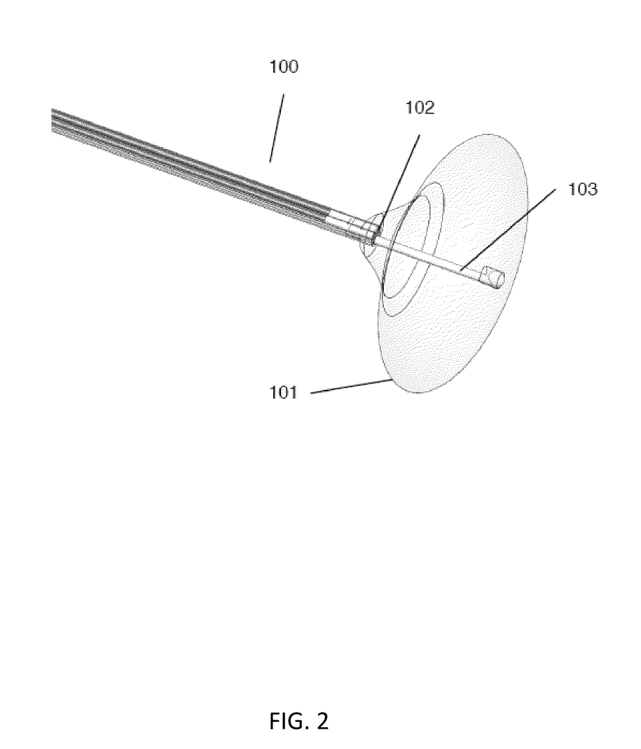

In reference to FIG. 1 and FIG. 2, in some embodiments, the visualization catheter 100 may include a main body 104 having one or more lumens. In some embodiments, the catheter body 104 may include an outer tube 106 and an inner tube 108 which define the one or more lumens of the catheter 100. The inner tube 108 may be semi-rigid to provide structure and support to the balloon 101 when the balloon 101 is in a deflated state. The inner tube 108 may also aid in navigation of the visualization catheter 100 either by housing a guide wire or pull-wire common to many catheter designs.

In some embodiments, the visualization catheter 100 of the present disclosure includes a balloon 101 disposed about a distal region of the visualization catheter. In some embodiments, since blood absorbs the illumination and fluorescence wavelengths, the balloon 101 may be used displace blood from the tissue surface. To do so, the balloon 101 may be expandable and compliant to seat well within the anatomy. In some embodiments, the balloon 101 may be made of a non-compliant material, but with variable sizes and shapes to fit into desired anatomy. In some embodiments, the balloon 101 may be constructed of a stronger material, such as polyurethane, to be less compliant or non-compliant. The balloon 101 may be of any shape that would best conform to various anatomic structures. In some embodiments, the balloon may be a round balloon, may have a flatter balloon shape than round, such as, for example, “lollipop” or effectively dome, bell, or cone shaped or pan-like, to increase the visible surface area. In some embodiments, the balloon can have a bell shape such that the narrower, distal portion may be advanced into an orifice and the wider, proximal portion of the balloon can oppose tissue at the base of the orifice while maximizing surface contact. In some embodiments, an anatomically conforming, compliant balloon may have a bell shaped balloon design, which can accommodate some of the bell shaped balloon into the orifice or ostium of the vein, while the broader base of the bell shaped balloon or the proximal end of the bell shaped balloon makes secure contact with the left atrial wall.

This may be useful in an orthogonal position of the balloon to tissue. In addition or alternatively to shapes, the balloon size can be scaled for improved maneuverability. In some embodiments, the balloon 101 may be configured to seal well against a flat, atrial wall anatomy, such as when AF lesions are created inside an atrial body.

The balloon 101 may also be constructed of a material that is optically clear in at least the relevant wavelengths for one or both illumination of the tissue (such as, myocardium) and fluorescence. In some embodiments, the balloon 101 may be optically transparent in the UV range of 330 nm to 370 nm. In some embodiments, the balloon 101 is optically clear from 330 nm to 370 nm for UV illumination and from 400 nm to 500 nm for the fluorescence wavelengths. Suitable UV-transparent materials for the balloon 101 include, but are not limited to, silicone and urethane.

The balloon 101 is moveable between a collapsed or deflated state for navigation of the balloon 101 to or from the treatment site to an expanded or inflated state at the treatment site, especially if delivered within an introducer sheath. To move the balloon 101 from a collapsed state to an expanded state, a fluid may be added to the balloon 101. The balloon may be moved from an expanded state to a collapsed state by withdrawing fluid from the balloon 101. The medium used to inflate the balloon 101 may also be optically transparent and yet ideally be fluoroscopically opaque for navigation purposes. Suitable inflation medium include, but are not limited to, Deuterium (heavy water) and CO₂, which meet both requirements. The medium may also be gas, such as nitrogen or carbon dioxide, or fluid such as saline or deionized water.

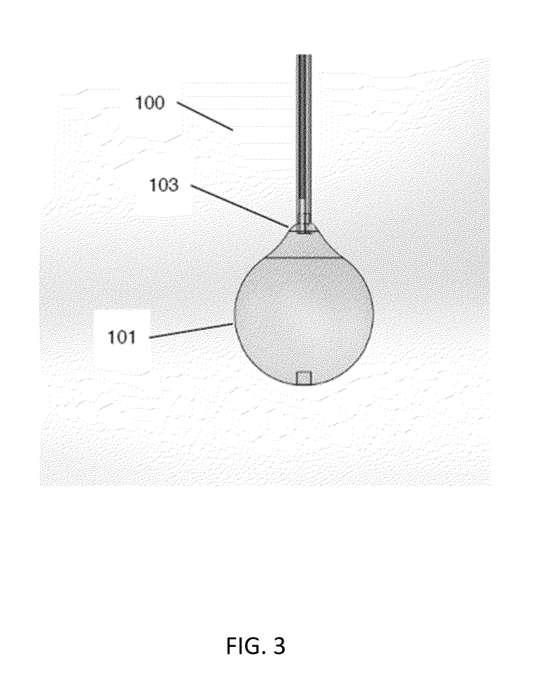

In some embodiments, the balloon 101 may be supported by a support assembly 103. In some embodiments, the balloon 101 attaches to the distal end of the catheter body 104 at its proximal end and to the distal end of the support assembly 103 at its distal end. The support assembly 103 may be fixed or may be retractable. The support assembly 103 may be permanently attached to the balloon 101. In some embodiments, the support assembly may detachably engage the balloon 101. In some embodiments, the balloon may include a receptacle or similar structure on its inner surface to facilitate detachable engagement between the support assembly 103 and the balloon 101. In reference to FIG. 3, the support assembly 103 may be retracted thus improving the field of view through the balloon 101. In some embodiments, the inner tube 108 extends beyond the outer tube 106 to form a support assembly 103. In some embodiments, the support assembly 103 may be independent of the inner tube 108. In some embodiments, the support from the inner tube 108 may prevent collapse of the balloon 101 and enhance both insertion of the balloon 101 into the body and subsequent navigation to the treatment site. Additionally or alternatively, the balloon may be supported by a guiding catheter or sheath.

The support assembly 103 may be designed to be gentle on the material of balloon 101 and yet provide enough column strength to provide sufficient support to enable the balloon to keep its shape within the introducer sheath. If the shape of the balloon 101 is not controlled, the balloon may become tangled upon itself and get stuck in the sheath. Forcing the balloon 101 in this situation can cause the balloon to tear.

There are various options to keep the support assembly and the balloon compatible. In some embodiments, the shape of the support assembly 103 may be made benign to the material of the balloon. For example, blunt or rounded or coil-shaped tip at the distal end of the support assembly 103 are but a few examples of an atraumatic distal end of the support assembly 101. Alternatively or additionally, the material of the balloon 101 may be reinforced at the support assembly interface. In some embodiments, wall thickness of the balloon material may be increased at that location or a protective material may be added. In some embodiments, the interface may not be optically clear in the relevant wavelengths in the case of the fully-extended support assembly.

In some embodiments, the balloon 101 may be a closed balloon. To contain fluid inside the balloon 101, the balloon 101 may have a closed end. Embodiments with a closed balloon may include, but are not limited to, diagnostic catheters with capability to visualize distal tissue because the member displaces blood and provides an optically transparent medium within the enclosed space. Other closed-member embodiments could also be therapeutic catheters where the therapy agent either passes through the inflation medium, such as laser ablation energy or light for photodynamic therapy, or is located on the outside of the balloon 101, such as a radiofrequency ablation electrode, or delivered through the closed balloon to the tissue, such as, injection of sclerosing agent, for example, ethanol.

In some embodiments, an open end balloon catheter may be employed to allow instruments or materials to pass through the balloon. In some embodiment's embodiments, the balloon is open at the distal end, but may be inverted to bond with the support assembly 103. To contain fluid inside the balloon 101, the balloon 101 may form a seal with the support assembly 103. In some embodiments, instruments or objects may be passed through the visualization catheter (such as through a lumen extending through the catheter body 104 and the support assembly 103) outside the balloon 101.

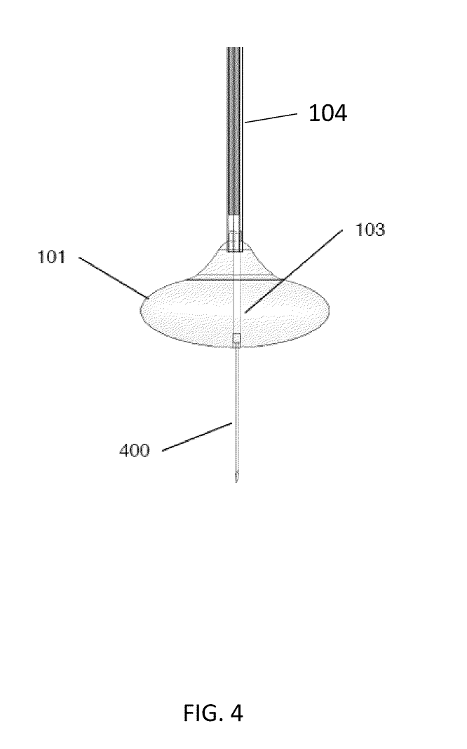

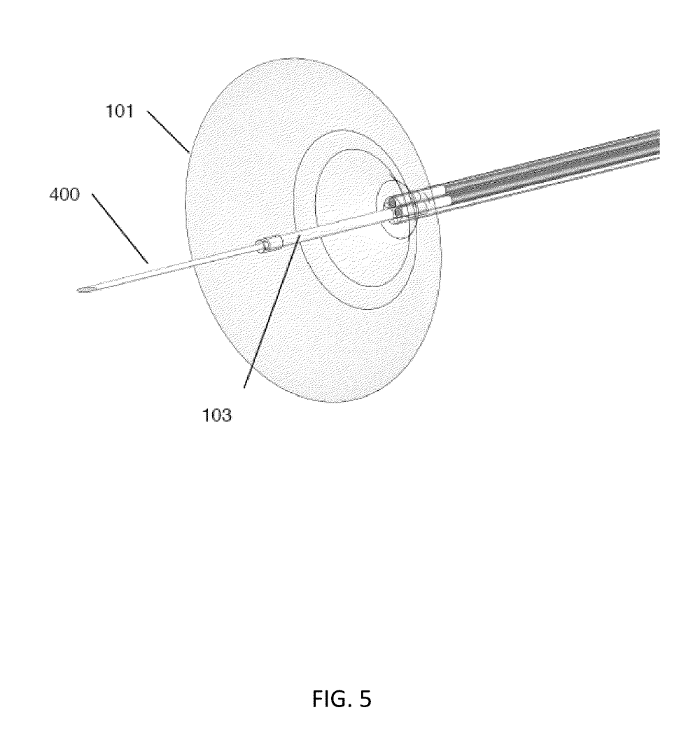

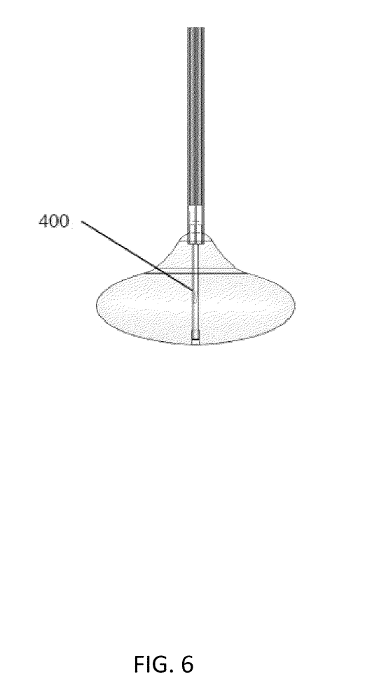

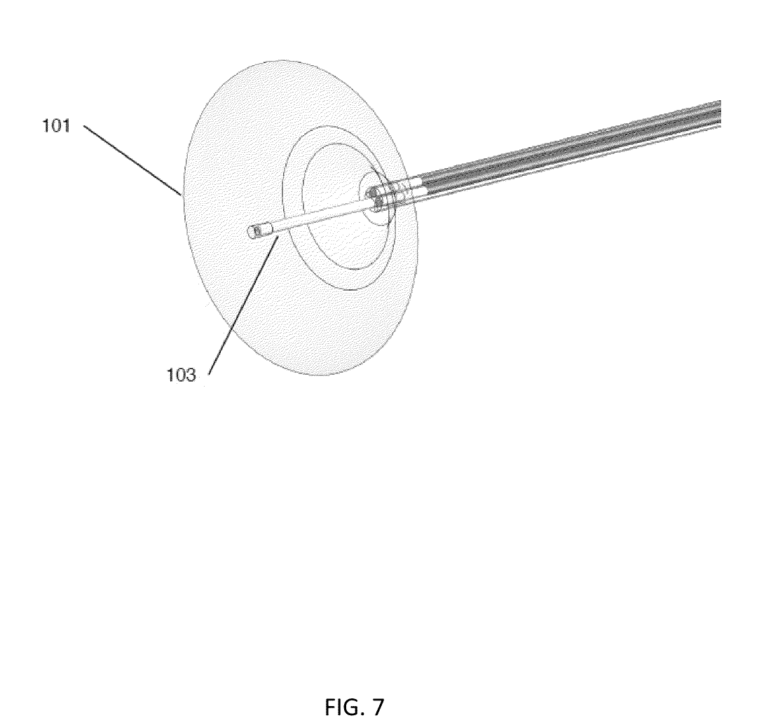

In reference to FIG. 4, FIG. 5, FIG. 6 and FIG. 7, in some embodiments, the visualization catheter 100 of the present may include an open end balloon 101 for assisting in transseptal access procedures. In some embodiments, a needle may be passed through a lumen of the catheter body 104 and the support assembly 103 past the distal end of the balloon 101 for making a transeptal puncture to gain access to the left side of the heart, while visualization is provided to the physician, as is described. Accordingly, physicians would be allowed to see the structures of the atrial septum, such as the foramen ovalis to visualize whether the needle 400 was crossing the septum at a safe location. In addition to the needle 400, catheters or other tools could pass through the lumen to enter into the other (i.e. left) side of the heart, for diagnostic or therapeutic procedures.

In some embodiments, the visualization catheter 101 may only include an outer tube 106 so the distal tip of the balloon 101 remains unsupported.

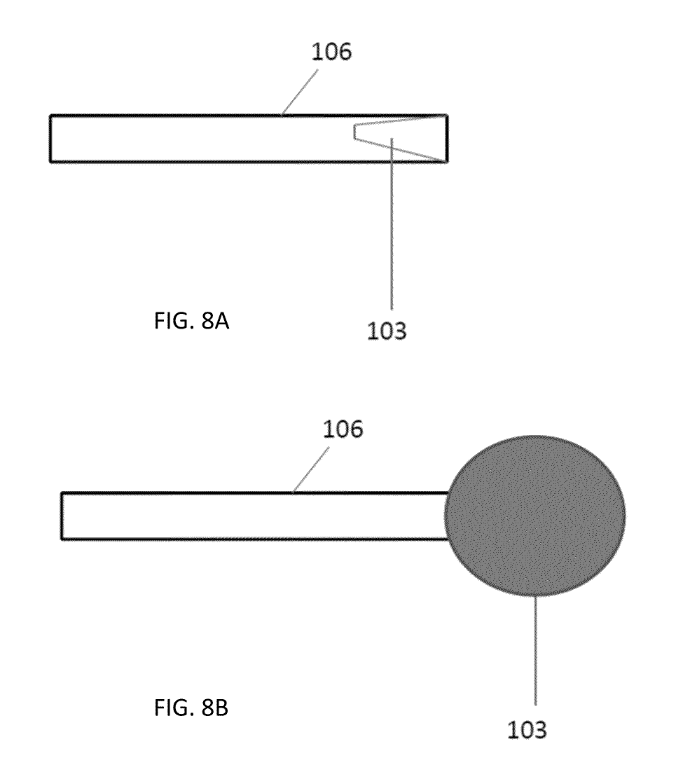

In some embodiments, the balloon 101 may be inverted into the lumen of the catheter 100 for delivery to a site of interest, as shown in FIG. 8A, and then everted with positive pressure to displace the blood, as shown in FIG. 8B. In some embodiments, the pressurized fluid in the balloon 101 may provide support for the balloon instead of or in addition to the support assembly 103.

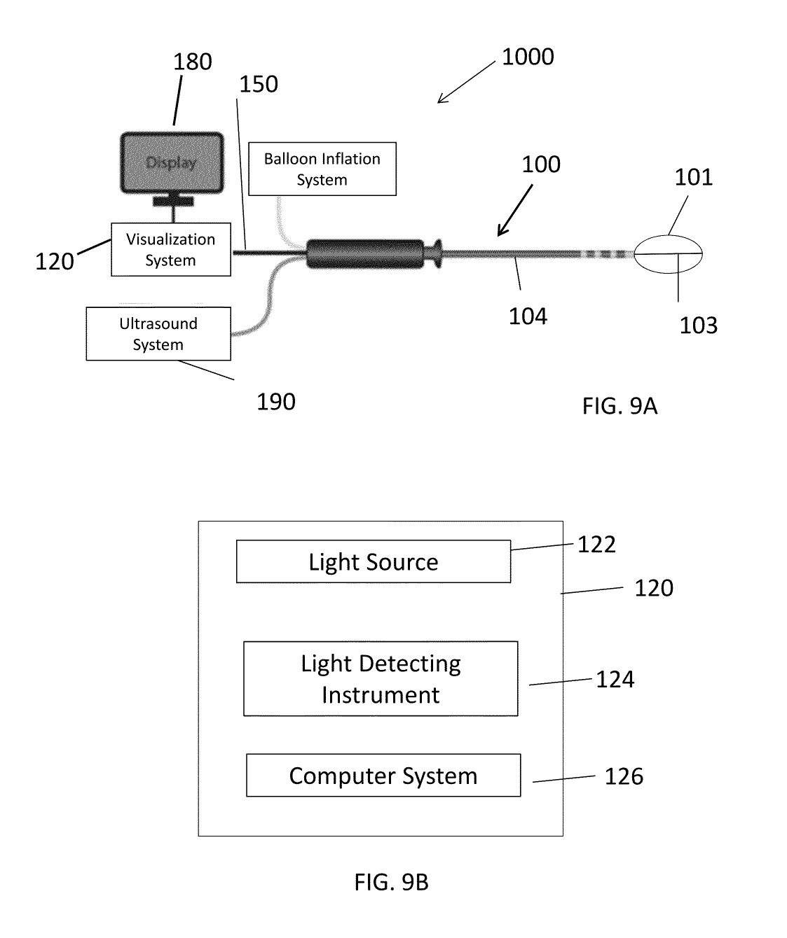

In reference to FIG. 9A, the catheter 100 is part of a diagnostic system 1000, which may also include a visualization system 120 for visualizing tissue. As shown in FIG. 9B, the visualization system 120 may include a light source 122, a light detecting instrument 124, and a computer system 126.

In some embodiments, the light source 122 may have an output wavelength within the target fluorophore (NADH, in some embodiments) absorption range in order to induce fluorescence in healthy myocardial cells. In some embodiments, the light source 122 is a solid-state laser that can generate UV light to excite NADH fluorescence. In some embodiments, the wavelength may be about 355 nm or 355 nm+/−30 nm. In some embodiments, the light source 122 can be a UV laser. Laser-generated UV light may provide much more power for illumination and may be more efficiently coupled into a fiber-based illumination system, as is used in some embodiments of the catheter. In some embodiments, the instant system can use a laser with adjustable power up to 150 mW.

The wavelength range on the light source 122 may be bounded by the anatomy of interest, a user specifically choosing a wavelength that causes maximum NADH fluorescence without exciting excessive fluorescence of collagen, which exhibits an absorption peak at only slightly shorter wavelengths. In some embodiments, the light source 122 has a wavelength from 300 nm to 400 nm. In some embodiments, the light source 122 has a wavelength from 330 nm to 370 nm. In some embodiments, the light source 122 has a wavelength from 330 nm to 355 nm. In some embodiments, a narrow-band 355 nm source may be used. The output power of the light source 122 may be high enough to produce a recoverable tissue fluorescence signature, yet not so high as to induce cellular damage. The light source 122 may be coupled to an optical fiber to deliver light to the balloon 101.

In some embodiments, the light detecting instrument 124 may comprise a camera connected to the computer system 126 for analysis and viewing of tissue fluorescence. In some embodiments, the camera may have high quantum efficiency for wavelengths corresponding to NADH fluorescence. One such camera is an Andor iXon DV860. The light detecting instrument 124 may be coupled to an imaging bundle that can be extended into the catheter 100 for visualization of tissue. In some embodiments, the imaging bundle for light detecting and the optical fiber for illumination may be combined. An optical bandpass filter of between 435 nm and 485 nm, in some embodiments, of 460 nm, may be inserted between the imaging bundle and the camera to block light outside of the NADH fluorescence emission band. In some embodiments, other optical bandpass filters may be inserted between the imaging bundle and the camera to block light outside of the NADH fluorescence emission band selected according to the peak fluorescence of the tissue being imaged.

In some embodiments, the light detecting instrument 124 may be a CCD (charge-coupled device) camera. In some embodiments, the light detecting instrument 124 may be selected so it is capable of collecting as many photons as possible and that contributes minimal noise to the image. Usually for fluorescence imaging of live cells, CCD cameras should have a quantum efficiency at about 460 nm of at least between 50-70%, indicating that 30-50% of photons will be disregarded. In some embodiments, the camera has quantum efficiency at 460 nm of about 90%. The camera may have a sample rate of 80 KHz. In some embodiments, the light detecting instrument 124 may have a readout noise of 8 e- (electrons) or less. In some embodiments, the light detecting instrument 124 has a minimum readout noise of 3 e-. Other light measuring instruments may be used in the systems and methods of the present disclosure.

The optical fiber 150 can deliver the gathered light to a long pass filter that blocks the reflected excitation wavelength of 355 nm, but passes the fluoresced light that is emitted from the tissue at wavelengths above the cutoff of the filter. The filtered light from the tissue can then be captured and analyzed by a high-sensitivity light detecting instrument 124. The computer system 126 acquires the information from the light detecting instrument 124 and displays it to the physician. The computer 126 can also provide several additional functions including control over the light source 122, control over the light detecting instrument 124, and execution of application specific software.

In some embodiments, the digital image that is produced by analyzing the light data may be used to do the 2D and 3D reconstruction of the lesion, showing size, shape, and any other characteristics necessary for analysis. In some embodiments, the image bundle may be connected to the light detecting instrument 124, which may generate a digital image of the lesion being examined from NADH fluorescence (fNADH), which can be displayed on the display 180. In some embodiment, these images can be displayed to the user in real time. The images can be analyzed by using software to obtain real-time details (e.g. intensity or radiated energy in a specific site of the image) to help the user to determine whether further intervention is necessary or desirable. In some embodiments, the NADH fluorescence may be conveyed directly to the computer system 126. In some embodiments, the optical data acquired by the light detecting instrument 124 can be analyzed to provide information about lesions during and after ablation including, but not limited to lesion depth and lesion size.

In some embodiments, the optical components (light source, the light detecting instrument or both) may be housed inside the balloon 101 and may communicate with an external computer system. In some embodiments, these components may be disposed on the inner tube 108, which may able to move independently of the outer tube 106 and the support assembly 103. As the inner tube 108 retracts from a fully extended position, it may move the optical components further from the target tissue to enable the field of view to expand to increase what the physician can see. In some embodiments, inflation of balloon without support assembly 103 can achieve the same result. In some embodiments, the optical components may be disposed on the support assembly 103.

In some embodiments, such as shown in FIG. 1, a light source/camera support assembly 103 may be disposed at the distal end of the outer tube 108 for positioning optical elements, such as a camera and a light source, inside the balloon. Positioning the light source inside the balloon may supplement the external light source or may eliminate the need for an external light source. Moreover, by putting the light sources within the balloon 101, wider angles of illumination may be achieved than when using a fiber bundle. The camera may be any image sensor that can convert an optical image or light signal into an electronic signal. In some embodiments, the camera is a miniature CMOS image sensor with a lens, and with or without a filter to choose a specific wavelength or set of wavelengths to record. In some embodiments, the camera is a CCD camera or other image sensors that can convert an optical image into an electronic signal. The camera may transmit its signal via wires to an image processor and video terminal for the physician to see. In some embodiments, the camera may have wireless communication capabilities for communication with external devices. The light source may be a light emitting diode (LED) of suitable wavelength. In some embodiments, the LED will have a wavelength in the UV range to cause the NADH fluorescence. In some embodiments, different wavelengths including white light for multicolor illumination are possible by choosing the LED of the appropriate wavelength. By way of a non-limiting example, suitable LEDs for UV applications would include those with wavelengths of 300 nm to 400 nm, while suitable LEDs for visible or white light applications would include those with color temperature ranges from 2000K to 8000K.

In some embodiments, the system 1000 of the present disclosure may further include an ultrasound system 190. The catheter 100 may be equipped with ultrasound transducers in communication with the ultrasound system. In some embodiments, the ultrasound may show tissue depths, which in combination with the metabolic activity or the depth of lesion may be used to determine if a lesion is in fact transmural or not.

In some embodiments, the diagnostic system 1000 of the present disclosure may include an ablation therapy system. The ablation therapy system may include one or more energy sources that can generate radiofrequency (RF) energy, microwave energy, electrical energy, electromagnetic energy, cryoenergy, laser energy, ultrasound energy, acoustic energy, chemical energy, thermal energy, or any other type of energy that can be used to ablate tissue. In some embodiments, the ablation therapy may be delivered using a separate ablation catheter. In some embodiments, the ablation therapy can be delivered using the catheter 100 of the present disclosure. In some embodiments, one or more electrodes may be painted on the balloon 101 and may be connected to the ablation therapy system to deliver ablation energy to tissue. The electrodes may be disposed on a distal face of the balloon, or may be disposed both on the distal face and side walls of the balloon. The electrodes may be connected to the ablation system for delivering ablation energy to tissue in contact with the balloon.

In some embodiments, the system 1000 may also include an irrigation system. In some embodiments, the system 100 may also include a navigation system for locating and navigating the catheter 100. In some embodiments, the catheter 100 may include one or more electromagnetic location sensors in communication with the navigation system. In some embodiments, the electromagnetic location sensors may be used to locate the tip of the catheter in the navigation system. The sensor picks up electromagnetic energy from a source location and computes location through triangulation or other means. In some embodiments the catheter 100 comprises more than one transducer adapted to render a position of the catheter body 104 and a curvature of the catheter body on a navigation system display. In some embodiments, the navigation system may include one or more magnets and alterations in the magnetic field produced by the magnets on the electromagnetic sensors can deflect the tip of catheters to the desired direction. Other navigation systems may also be employed, including manual navigation.

The visualization catheter of the present disclosure may be used in a variety of procedures, such as for example, transseptal procedures, ablation lesion mapping, ablation lesion formation, and photodynamic therapy, among others.

In some embodiments, to treat arrhythmias that originate in the left atrium or left ventricle, access to the left side of the heart is required. The left side of the heart may be accessed through a transseptal puncture. In operation, the visualization catheter may be advanced into the right atrium via either the inferior or superior vena cava, after being inserted into either a femoral or potentially brachiocephalic vein, respectively. Once in the right atrium, the catheter may be pressed up against the fossa ovalis. Next, a needle or another puncturing instrument may be advanced through the lumen of the visualization catheter to puncture a hole through the septum. The visualization catheter or a different catheter may be advanced through the puncture into the left side of the heart. The procedure may be visualized using the visualization system 120. In some embodiments, direct visualization may be employed.

In some embodiments, the visualization catheter 100 of the present disclosure is used for ablation lesion mapping. In some embodiments, the mapped lesions may be from a previous procedure. In some embodiments, the mapping may be in real time in combination with ablation procedure. In some embodiments, the visualization catheter 100 may be used for diagnostics, in combination with a separate ablation catheter. In some embodiments, the visualization catheter 100 may be designed for both for the therapeutic function to deliver ablation energy to tissue to form lesions and for the diagnostic function of visualizing such lesions.

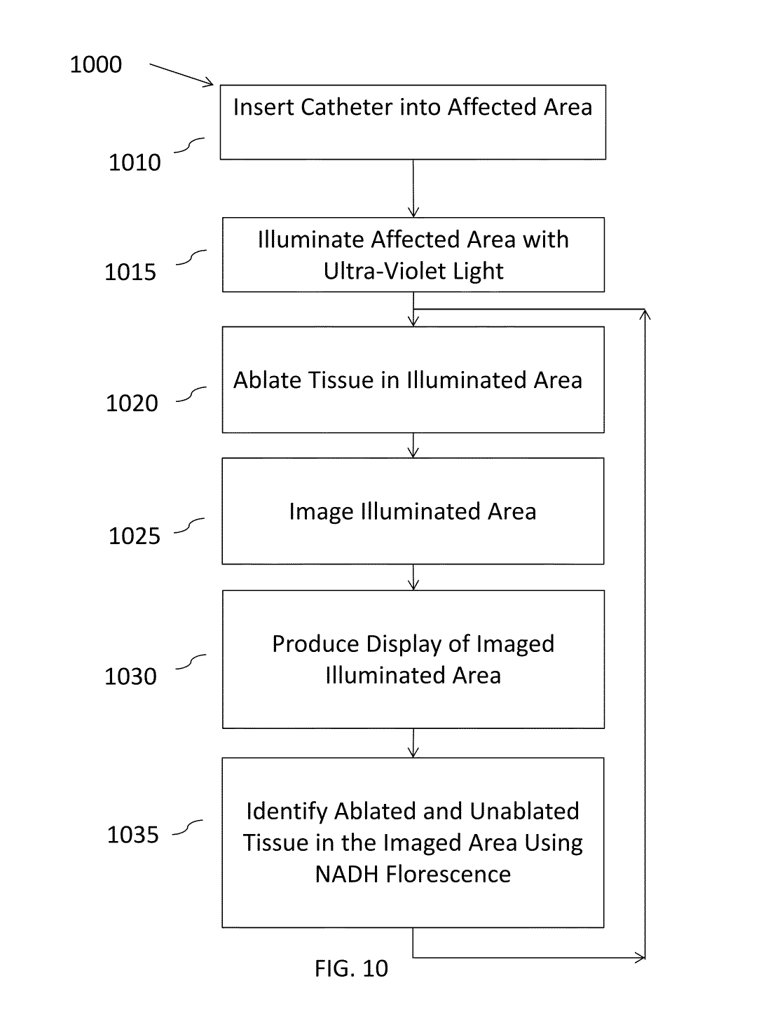

FIG. 10 further illustrates operation of the diagnostic system 1000 of the present disclosure. Initially, the catheter 100 is inserted into the area of heart tissue affected by the atrial fibrillation, such as the pulmonary vein, left atrium, left atrial junction or another area of the heart (step 1010). Blood may be removed from the visual field, for example, by irrigation or using the balloon. The affected area may be illuminated by ultra-violet light reflected from the light source (step 1015). Tissue in the illuminated area may be ablated (step 1020), either before, after, or during illumination. Either point-to-point RF ablation or cryoablation or laser or other known ablation procedures may be employed using the systems of the present disclosure.

The illuminated area may be imaged by receiving the light from the tissue with the camera (step 1025). In some embodiments, the methods of the present disclosure rely on imaging of the fluorescence emission of NADH, which is a reduced form of nicotinamide adenine dinucleotide (NAD+). NAD+ is a coenzyme that plays important roles in the aerobic metabolic redox reactions of all living cells. It acts as an oxidizing agent by accepting electrons from the citric acid cycle (tricarboxylic acid cycle), which occurs in the mitochondrion. By this process, NAD+ is thus reduced to NADH. NADH and NAD+ are most abundant in the respiratory unit of the cell, the mitochondria, but are also present in the cytoplasm. NADH is an electron and proton donor in mitochondria to regulate the metabolism of the cell and to participate in many biological processes including DNA repair and transcription.

By measuring the UV-induced fluorescence of tissue, it is possible to learn about the biochemical state of the tissue. NADH fluorescence has been studied for its use in monitoring cell metabolic activities and cell death. Several studies in vitro and in vivo investigated the potential of using NADH fluorescence intensity as an intrinsic biomarker of cell death (either apoptosis or necrosis) monitoring. Once NADH is released from the mitochondria of damaged cells or converted to its oxidized form (NAD+), its fluorescence markedly declines, thus making it very useful in the differentiation of a healthy tissue from a damaged tissue. NADH can accumulate in the cell during ischemic states when oxygen is not available, increasing the fluorescent intensity. However, NADH presence disappears all together in the case of a dead cell. The following table summarizes the different states of relative intensity due to NADH fluorescence:

| | | Relative Changes of Auto- |
|---|---|---|
| Cellular State | NADH Presence | fluorescense intensity |
| Metabolically Active | Normal | Baseline |
| Metabolically Active but | Increased due | Increased |
| Impaired (Ischemia) | to Hypoxia | |
| Metabolically Inactive | None | Full Attenuation |
| (Necrotic) | | |

Still referring to FIG. 10, while both NAD+ and NADH absorb UV light quite readily, NADH is autofluorescent in response to UV excitation whereas NAD+ is not. NADH has a UV excitation peak of about 340 to about 360 nm and an emission peak of about 460 nm. In some embodiments, the methods of the present disclosure may employ excitation wavelengths between about 330 nm to about 370 nm. With the proper instrumentation, it is thus possible to image the emission wavelengths as a real-time measure of hypoxia as well as necrotic tissue within a region of interest. Furthermore, in some embodiments, a relative metric can be realized with a grayscale rendering proportionate to NADH fluorescence.

Under hypoxic conditions, the oxygen levels decline. The subsequent fNADH emission signal may increase in intensity indicating an excess of mitochondrial NADH. If hypoxia is left unchecked, full attenuation of the signal will ultimately occur as the affected cells along with their mitochondria die. High contrast in NADH levels may be used to identify the perimeter of terminally damaged ablated tissue.

To initiate fluorescence imaging, NADH may be excited by the UV light from the light source, such as a UV laser. NADH in the tissue specimen absorbs the excitation wavelengths of light and emits longer wavelengths of light. The emission light may be collected and passed back to the light detecting instrument, and a display of the imaged illuminated area may be produced on a display (step 1030), which is used to identify the ablated and unablated tissue in the imaged area based on the amount of NADH florescence (step 1035). For example, the sites of complete ablation may appear as completely dark area due to lack of fluorescence. Accordingly, the areas of ablation may appear markedly darker when compared to the surrounding unablated myocardium, which has a lighter appearance. This feature may enhance the ability to detect the ablated areas by providing marked contrast to the healthy tissue and even more contrast at the border zone between ablated and healthy tissue. This border area is the edematous and ischemic tissue in which NADH fluorescence becomes bright white upon imaging. The border zone creates a halo appearance around the ablated central tissue.

The process may then be repeated by returning to the ablation step, if necessary, to ablate additional tissue. It should be recognized that although FIG. 10 illustrates the steps being performed sequentially, many of the steps may be performed simultaneously or nearly simultaneously, or in a different order than shown in FIG. 10. For example, the ablation, imaging and display can occur at the same time, and the identification of the ablated and unablated tissue can occur while ablating the tissue.

The visualization catheter 100 of the present disclosure may also be used in variety of light sensitive applications. One such application may be photodynamic therapy (PDT). PDT is often used for cancer treatment, wherein a drug is delivered systemically, but remains inert until exposed to UV light or a specific wavelength of light. Thus by shining light on the target areas (i.e. vasculature within tumors), the drug can be selectively activated in the target locations while sparing the drug's effect on the rest of the body. Other applications may include renal artery denervation, which is used in the treatment of hypertension. The denervating drug could be deployed via the balloon, or systemically, and activated by the light energy from the catheter assembly. Other applications for which the visualization catheters 100 of the present disclosure can be used include tissue ablation. For example, light sources, such as laser, may be used to direct energy into a tumor to ablate the tumor. Benign Prostatic Hyperplasia (BPH) may also be treated by ablation, which provides means for achieving deep and controlled depth of penetration of light energy into prostatic tissue. Ablation may also be used in heart tissue or normal parts of the heart to destroy abnormal electrical pathways that, for example, are contributing to a cardiac arrhythmia. Ablation also presents a minimally invasive alternative for the treatment of varicose veins.

The foregoing disclosure has been set forth merely to illustrate various non-limiting embodiments of the present disclosure and is not intended to be limiting. Since modifications of the disclosed embodiments incorporating the spirit and substance of the disclosure may occur to persons skilled in the art, the presently disclosed embodiments should be construed to include everything within the scope of the appended claims and equivalents thereof. All references cited in this application are incorporated herein by reference in their entireties.

What is claimed is:

1) A catheter for visualizing ablated tissue comprising:
a catheter body;
a support assembly extending past a distal end of the catheter body, the support assembly having a lumen therethrough; and
a balloon having a proximal end and a distal end, wherein the proximal end of the balloon is attached to the catheter body and the distal end of the balloon is attached to the support assembly, the balloon having an opening at the distal end in alignment with the lumen of the support assembly to provide a continuous path from the catheter body to outside of the balloon.

2) The catheter of claim 1 wherein the support assembly is retractable in and out of the catheter.

3) The catheter of claim 1 further comprising one or more optical fibers extending into the balloon to deliver light to and from the balloon.

4) The catheter of claim 1 further comprising a light source and a light detecting instrument housed inside the balloon.

5) The catheter of claim 1 wherein the balloon is bell shaped to fit into a pulmonary vein.

6) The catheter of claim 1 wherein the balloon is made of a compliant ultraviolet (UV) light transparent material.

7) The catheter of claim 1 wherein one or more ablation electrodes are disposed on the balloon to deliver ablation energy to tissue.

8) The catheter of claim 1 wherein the distal tip is configured to deliver ablation energy to the tissue, the ablation energy being selected from a group consisting of radiofrequency (RF) energy, microwave energy, electrical energy, electromagnetic energy, cryoenergy, laser energy, ultrasound energy, acoustic energy, chemical energy, thermal energy and combinations thereof.

9) The catheter of claim 1 further comprising an ultrasound transducer.

10) A system for visualizing ablated tissue comprising:
a catheter comprising a catheter body; a support assembly extending past a distal end of the catheter body, the support assembly having a lumen therethrough; and a balloon having a proximal end and a distal end, wherein the proximal end of the balloon is attached to the catheter body and the distal end of the balloon is attached to the support assembly, the balloon having an opening at the distal end in alignment with the lumen of the support assembly to provide a continuous path from the catheter body to outside of the balloon;
a light source; and
a light detecting instrument.

11) The system of claim 10 further comprising one or more optical fibers in communication with the light source and the light detecting instrument and extending through the catheter body into the balloon for illuminating tissue outside the distal tip and collecting and relaying light energy reflected from the tissue to the light detecting instrument.

12) The system of claim 10 wherein the light source and the light detecting instrument are housed inside the balloon.

13) The system of claim 10 wherein the support assembly is retractable in and out of the catheter.

14) The system of claim 10 wherein the light source emits light having a wavelength between about 300 nm and about 400 nm.

15) The system of claim 10 wherein one or more ablation electrodes are disposed on the balloon to deliver ablation energy to tissue.

16) The system of claim 10 further comprising a source of ablation energy in communication with the balloon to deliver ablation energy to the tissue, the ablation energy being selected from a group consisting of radiofrequency (RF) energy, microwave energy, electrical energy, electromagnetic energy, cryoenergy, laser energy, ultrasound energy, acoustic energy, chemical energy, thermal energy and combinations thereof.

17) A method for transseptal access to a left atrium comprising:
advancing a catheter to a right atrium, the catheter comprising a catheter comprising a catheter body; a support assembly extending past a distal end of the catheter body, the support assembly having a lumen therethrough; a balloon having a proximal end and a distal end, wherein the proximal end of the balloon is attached to the catheter body and the distal end of the balloon is attached to the support assembly, the balloon having an opening at the distal end in alignment with the lumen of the support assembly to provide a continuous path from the catheter body to outside of the balloon; and a camera;
delivering a puncturing instrument through the path in the catheter body to outside the balloon; and
under visualization with the camera, pushing the puncturing instrument against the fossa ovalis to make an access hole into the left atrium.

18) The method of claim 17 wherein the camera is housed within the balloon.

19) The method of claim 17 where the camera is external to the catheter and one or more optical fibers extend from the camera to the balloon to visualize tissue outside the balloon.

20) A method for ablation mapping comprising:
advancing a catheter to a cardiac tissue in need of ablation mapping, the catheter comprising a catheter comprising a catheter body; a support assembly extending past a distal end of the catheter body, the support assembly having a lumen therethrough; a balloon having a proximal end and a distal end, wherein the proximal end of the balloon is attached to the catheter body and the distal end of the balloon is attached to the support assembly, the balloon having an opening at the distal end in alignment with the lumen of the support assembly to provide a continuous path from the catheter body to outside of the balloon; and a camera;
exciting nicotinamide adenine dinucleotide hydrogen (NADH) in an area of the cardiac tissue;
collecting light reflected from the cardiac tissue and directing the collected light to a light detecting instrument;
imaging the area of the cardiac tissue to detect NADH fluorescence of the area of the cardiac tissue; and
producing a display of the imaged, illuminated cardiac tissue, the display illustrating the ablated cardiac tissue as having less fluorescence than non-ablated cardiac tissue.
